(19) United States
(12) Patent Application Publication
(10) Pub. No.: US 2004/0133082 A1
(43) Pub. Date: Jul. 8, 2004

(54) System for medically assisting the occupants of a motor vehicle

(76) Inventors: Klaus Abraham-Fuchs, Erlangen (DE); Tilo Christ, Erlangen (DE); Markus Schmidt, Nuernberg (DE); Volker Schmidt, Erlangen (DE); Siegfried Schneider, Erlangen (DE); Sven Tiffe, Wien (AT)

Correspondence Address: HARNESS, DICKEY & PIERCE, P.L.C., P.O.BOX 8910, RESTON, VA, 20195, US

(21) Appl. No.: 10/689,952

(22) Filed: Oct. 22, 2003

(30) Foreign Application Priority Data
Oct. 23, 2002 (DE) 10249415.0

(51) Int. Cl. A61B 5/00

(52) U.S. Cl. 600/300; 600/481; 180/271; 340/576; 604/403

(57) ABSTRACT

A motor vehicle includes an assistance device for assisting in a diagnosis, therapy and/or prophylactic treatment on a person who is in the vehicle. An electronic data processing device is for automatically actuating and/or interrogating the assistance device. The assistance device can automatically activate at least one device intended to prompt the person to perform an action, e.g. to take medicaments or to perform a physiological exercise. There can also be an identification device for identifying the person, in order to actuate and/or to interrogate the assistance device on the basis of the result of the identification of the person.

The present application hereby claims priority under 35 U.S.C. §119 on German patent application number DE 102 49 415.0 filed Oct. 23, 2002, the entire contents of which are hereby incorporated herein by reference.

### FIELD OF THE INVENTION

The invention generally relates to a system for medical therapy, diagnosis or preventative therapy for the occupants of a motor vehicle.

### BACKGROUND OF THE INVENTION

Systems for medical diagnosis or therapy in motor vehicles are known from ambulances, which are equipped with all the devices necessary for this purpose. In this case, medical care is provided exclusively for the patient and is initiated and performed by the ambulance team. It is brief in as much as ambulances are used only for transfer journeys to hospitals.

A system for medically treating a motor vehicle occupant is also already known from the written specification AU 98 78838 A. This document discloses a vaporizer for therapeutic oils which can be connected to the cigarette lighter in the vehicle. The treatment is thus not performed automatically, but rather must first be initiated by the occupant of the motor vehicle.

A similar apparatus is already known from the written specification U.S. Pat. No. 4,335,725, which discloses a therapeutic heat cushion for a seat. This is likewise connected to the cigarette lighter in the motor vehicle, and thus likewise first needs to be installed and started by the occupant.

### SUMMARY OF THE INVENTION

An object of an embodiment of the invention is to specify a system which gives or offers medical assistance to the occupants of a motor vehicle automatically without first needing to be initiated manually or by third parties.

An embodiment of the invention achieves an object by use of an apparatus which is integrated in a motor vehicle.

A concept of an embodiment of the invention is that the system automatically enters into interaction with the occupants by performing identification of the occupants, which is necessary for medical purposes, or asking the occupants to perform an activity on medical grounds. The request for the occupants to perform an activity can involve the occupant being asked to input data into the system, to remove and then to take medicaments or to perform physiological exercises on medical grounds. The advantage is obtained that the medical assistance can be provided on the basis of a possibly complex time or action plan which is stored in the system, that it is not overlooked on account of human forgetfulness and that initiating it does not require any effort. The identification also allows medications indicated specifically for the identified person to be administered, allows individual long-term profiles of physiological measured data to be recorded or allows an individually created physiological exercise program to be activated.

An embodiment of the invention is based on the insight that the motor vehicle, as the subject of daily use, provides a regularly available framework for medical measures which are to be taken daily. What is in mind here, in particular, is measures which need to be taken repeatedly over relatively long periods of time. Such tasks can be long-time medicament administration, the recurring measurement of physiological parameters for the person for the purposes of monitoring, or particular physiological exercises, suitable for the car environment, of an orthopedic or circulatory nature. The advantage obtained is that the person who is to be assisted is able to take necessary medical measures more or less incidentally during use of the motor vehicle without being burdened by them all too greatly or at unsuitable times. As such, time spent in the motor vehicle which otherwise pass unproductively are used for medical purposes.

One advantageous refinement of an embodiment of the invention involves the system ascertaining data about the present traffic and driving situation of the motor vehicle. Thus, measures can be initiated at times at which the driving or traffic burden on the driver or on other occupants allows this.

### BRIEF DESCRIPTION OF THE DRAWINGS

The present invention will become more fully understood from the detailed description of preferred embodiments given hereinbelow and the accompanying drawings, which are given by way of illustration only and thus are not limitative of the present invention, and wherein:

FIG. 1 shows the medical assistance system based on an embodiment of the invention with an interactive communication device,

FIG. 2 shows the medical assistance system based on an embodiment of the invention with apparatuses for medicament dispensing,

FIG. 3 shows the medical assistance system based on an embodiment of the invention with physiological sensors.

### DETAILED DESCRIPTION OF THE PREFERRED EMBODIMENTS

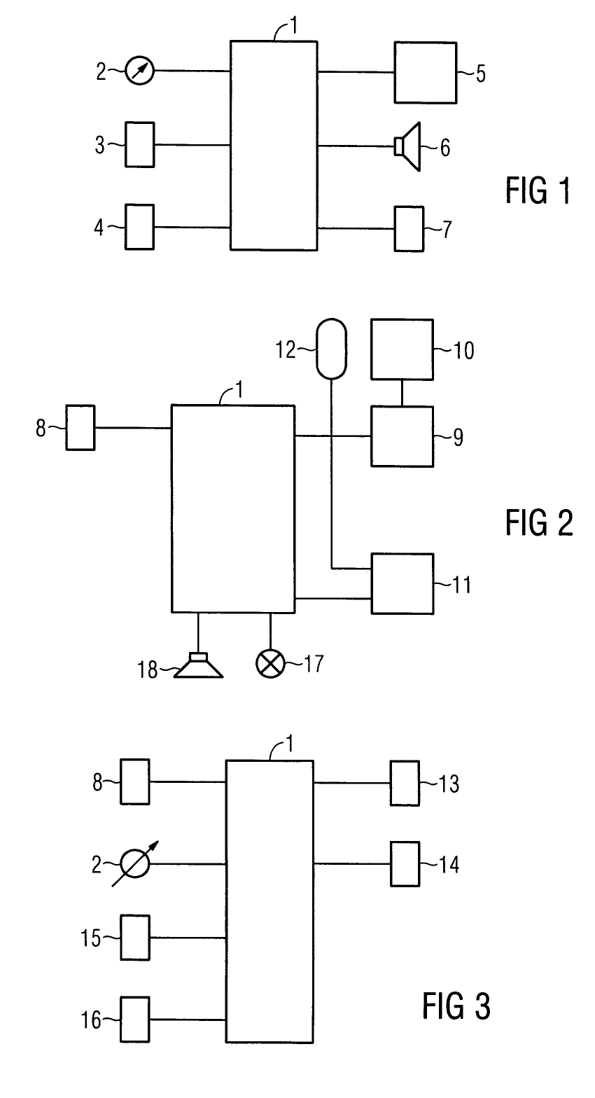

FIG. 1 schematically shows a medical system for assisting the occupants of a vehicle in line with an embodiment of the invention. A central part of the system is an electronic data processing device 1 which has a microprocessor, a suitable data store and input and output channels for connecting the further components of the system. The electronic data processing device 1 is installed permanently at a suitable location in the motor vehicle, e.g. in the central console or under one of the vehicle seats. The voltage supply is taken from the vehicle's on-board power supply, to which there is likewise a permanently installed electrical connection. If a voltage supply other than the on-board power supply is intended to be used, for example because the medical system requires a higher operating voltage, it is possible to use a separate voltage supply.

The medical system in FIG. 1 has interactive communication means 5, 6, 7 which it can use to provide the occupants of the vehicle with information and to receive inputs from these occupants. The interactive communication device includes a screen 5, which is of touch-sensitive design, i.e. is in the form of a “touchscreen”. For this purpose, the screen is connected via an input line and a further output line, which are both shown separately in FIG. 1. The touchscreen 5 is arranged such that it can easily be seen by the occupants of the vehicle, e.g. in or above the central console.

As a further communication device, the system in FIG. 1 has a loudspeaker 6 and a microphone 7. Depending on the available vehicle equipment, the loudspeaker 6 used can be a hands-free device which has already been installed or the loudspeaker for a car radio which is already present. Similarly, the microphone 7 used can be the microphone in a hands-free installation which has already been installed. The medical system is able to use the loudspeaker 6 to provide the occupants with audible messages, for example announcements which are stored or are generated by way of a voice generator, and it can receive instructions from the occupants via the microphone 7. If required, further input and output elements, including but not limited to e.g. monitor lamps, pushbuttons, etc. can be provided.

Since particularly the driver of a motor vehicle should not be substantially distracted during the journey, the medical system in FIG. 1 includes devices which it can use to recognize road traffic situations during which interaction with the driver or with the occupants is possible without danger. These can include, but are not limited to, e.g. parking brakes, stationary phases in queues or monotonous driving situations on little-frequented roads, etc.

To be able to recognize the respective traffic situation, the electronic data processing device 1 may be permanently connected to the motor vehicle's tachometer 2 and is therefore always informed about the respective current speed of travel. In addition, the system may be informed about the current position of the vehicle, about the direction of movement and about the speed by a GPS system 3 which likewise may be connected. Furthermore, the system may evaluate additional data about the current traffic situation, which it receives from a traffic control system and from communication channels on the radio via a receiver 4.

The system shown in FIG. 1 is programmed such that it asks the occupants to perform preventive physiological exercises. These can serve, by way of example, to combat back complaint, which is regarded as a national complaint anyway but which particularly affects people who frequently have to cover long distances in a motor vehicle. If it has recognized a suitable traffic situation, the system can spontaneously offer training exercises to prevent back complaint via the touchscreen 5 and/or the loudspeaker 6.

The occupants are then provided with the option of using the touchscreen 5 and/or the microphone 7 to reject or to confirm the performance of exercises. If they reject the performance of exercises, then the medical system offers exercises again after a particular waiting time as soon as it recognizes a suitable traffic situation again. If they confirm the performance of exercises, on the other hand, they are then provided with the opportunity to influence the start and course of the exercise program. They can select from a plurality of exercise programs which differ in type and intensity and in duration. To this end, the occupants interactively choose between various options, and they also input signals to provide notification about the completion of individual exercises or the desire to start a new exercise.

Depending on the training exercise, the electronic data processing device 1 can operate on the basis of an adjustable algorithm which adjusts itself to the occupants' training behavior. Adjustment then takes place firstly on the basis of the inputs from the occupants, and secondly additional information can be obtained by further sensors. Thus, by way of example, the occupants' body posture can be monitored using sensors integrated in the seats. Detection of the body posture and also additional measurement of the forces arising by the sensors provide the medical system with further information on the progress of training.

In addition, the type and scope of the exercises can be shaped individually for individual people if the medical system obtains information which allow identification of the occupants. The identification can be made by user inputs, for example. In addition, when the individual seats in the vehicle are recurrently used by the same occupants, exercises can be shaped for these seats and hence indirectly for the regular users of these seats. Also, information can be used which is stored in the electronic immobilizer chips in user-specific vehicle keys. Not least, it is possible to use biometric measurements, e.g. the fingerprint or the eye's iris, for identification purposes.

The use of body posture sensors in the vehicle seats also makes it possible to propose preventive exercises or posture corrections as soon as it is established that one of the occupants has maintained an incorrect body posture for a relatively long time. Exercises can also be proposed if the vehicle exceeds a particular traveling time, for example after two hours of traveling time in each case.

Besides the body posture, it is also possible for the correct performance of the exercises to be monitored, and correction advice can possibly be given. A further option for controlling the performance of the exercises is to record electromyograms, for example relating to the driver's manual contact with the vehicle's steering wheel. These can give an indication of the scope and intensity of the work by the muscles when performing the exercises.

One particular functionality of the medical system involves recognizing developing queue situations on the basis of the information received by the traffic control system receiver 4. In such situations or while stationary in the queue, the occupants are then asked to perform muscle pumping exercises in order to stimulate the circulatory system and thereby to ensure prevention of thrombosis formation. By way of assistance, breathing exercises can also be added. Not least, it is possible to assist back exercises and prevention of thrombosis by virtue of the medical system activating a seat heating system or a massage system integrated in the seats.

FIG. 2 schematically shows a further refinement of an embodiment of the invention which is likewise based on a central electronic data processing device 1. It is permanently connected to apparatuses for direct, metered dispensing of active agents.

First, it controls the medicaments dispenser 9, which is able to dispense particular medicaments in solid form which are supplied to it from the medicaments reservoir 10. The medicaments dispenser 9 can be in the form of a drawer from which the respective substance can be removed or in the form of a tube whose end dispenses tablets, for example, into a user's hand when the user actuates it. The medicaments reservoir 10 can store various medicaments in various administration forms, but, with regard to using it and taking them with as few problems as possible in the car, these will essentially be medicaments in tablet form.

In addition, a likewise permanently connected liquids dispenser is controlled which can directly dispense particular doses of various liquids from a liquid reservoir 12. The liquids dispenser 11 can work in a comparable manner to a drinks machine in that it fills a plastic cup with liquids and the plastic cup can then be removed by one of the vehicle occupants. The liquids can be therapeutic active agents. They can also be water offered to facilitate the taking of tablets from the medicaments dispenser 9 or to stabilize the vehicle occupant's balance of liquids.

The system in FIG. 2 also has a permanently connected sensor 8 which allows identification of the vehicle occupants. Identification can take place on a biometric basis, and also the aforementioned interactive options are available. Identification allows medicaments to be provided on a patient-specific basis.

The medical system shown in FIG. 2 is particularly suitable for therapy for patients who have to take regular medication or have to observe dietetic measures. In this regard, the system is particularly suitable for use in motor vehicles by people who recurrently use the vehicle on a daily basis for the regular journey to work or in another way. These periods of time can be used for the long-term regular administering of medicaments. This can simultaneously improve “therapy compliance”, that is to say willingness to take and punctuality in taking the medication, since there is a regular reminder of necessary therapeutic measures.

If the system is used to assist the taking of medicaments, these are metered and provided individually taking into account the time of day and the intervals at which they are taken. The logistic process is assisted by virtue of stocks which are running low in the reservoirs 10, 12 being signaled automatically. Depending on the desired degree of automation, stocks which are running low can be reordered by the system automatically, e.g. over the Internet. For this purpose there is an interface (which is not shown in FIG. 2 but is shown for the first time in FIG. 3) for data communications 16, preferably a mobile radio modem which can be used to set up Internet connections or other data links.

The data communication link can be used by the system not only to perform logistic tasks but also to connect to an electronic patient record, firstly in order to obtain information about the necessary medication from the patient record and secondly to be able to store information about the time and type of the medicaments taken by the patient in the patient record. In addition, all further physiological and other information can naturally be stored in the patient record. This allows the system to assist remote medicament monitoring. Not least, the data communication link can be used to send alarm signals in emergencies such as intolerance of medicaments or else traffic accidents.

The data communication link 16 can also be used to receive information. By way of example, data which the system ascertains can be taken as a basis for providing medical consultancy over the data communication link. This can involve behavioral measures, health-related information or physiotherapy exercises and posture exercises being transmitted. The system then notifies the vehicle occupants of these via the communication means 5, 6, 7. The consultancy can be provided either on the basis of situation using the data ascertained by the system or interactively using inputs by the occupants via the interactive communication means 5, 7. Finally, the data communication link 16 can also be used to receive information about the current road traffic situation.

The reservoirs 10, 12 are accommodated directly in the interior of the vehicle or else round about, e.g. in the trunk or under the hood, depending on the type and scope of the medication. Particularly the solids reservoir 10 is preferably fitted in the immediate vicinity of the medicaments dispenser 9 in order to facilitate transfer of the medicaments from the reservoir 10 to the dispenser 9. By way of example, it can be accommodated instead of a glove compartment or in a glove compartment or in the central console in the motor vehicle.

Since transfer of liquids between the liquids reservoir 12 and the liquids dispenser 11 is significantly easier to implement, the liquids reservoir 12 can readily be installed in the vehicle's trunk and can be connected to the liquids dispenser 11 by means of a liquids line. Accommodating it under the hood is normally unsuitable on account of the heat given off by the engine. The liquids dispenser 11 dispenses water, inter alia, which is provided at the same time as tablets for the purpose of taking the latter. This makes them easier to swallow, and it is also possible to dissolve medicaments provided in powder form in the water. Furthermore, it is additionally possible to dispense further, different liquids which themselves contain therapeutically active substances.

The identification sensor 8 allows the system to recognize particular occupants and to administer individual medications. In addition, the system can provide medicaments on the basis of the environmental situation. By way of example, the vehicle's ventilation system can have a pollen detector installed in it which informs the medical system about the occurrence of pollen at present. If it detects that there is a high pollen count, the system can automatically provide antiallergenic medicaments. In addition, the system can provide vitalizing substances, such as vitamin preparations or caffeine tablets, after a particular traveling time has elapsed, the traveling time having been ascertained by the system from tachometer information, for example.

If the system is intended to be used to implement dietary plans, the regularity of food intake can be monitored for individual occupants. It is also possible to give a reminder about the application of insulin, which in one particular embodiment of the system can be provided by the medicaments dispenser 9. In addition, it is also possible to input information about restaurants which are suitable for diabetics into the system, so that a link to a navigation system fitted in the car can be used to assist navigation to such restaurants.

Depending on the type of medicaments which are to be dispensed, the medicaments dispenser 9 and the liquids dispenser 11 can be in different forms. They are preferably installed in or on the console of the vehicle, where they can easily be seen by the vehicle occupants. Thus, it is probable that occupants will become aware of liquids or medicaments being provided themselves. However, the system is complemented by additional signal transmitters, an indicator LED 17 and an indicator buzzer 18, which it uses to indicate that medicaments are being provided or that particular times in the dietetic nutritional plan have arrived. In response to the prior generation of an indicator signal, it is also possible for the occupants of the motor vehicle to reject the indicated provision of a substance by the medicaments dispenser 9 or the liquids dispenser 11. This makes it possible to prevent substances from having to be put back or destroyed when they are not removed by the occupants.

The medicaments dispenser 9 and the liquids dispenser 11 are designed such that they register removal by one of the occupants. If substances provided are not removed, they can be disposed of or put back. This prevents operating faults in the two dispensers 9, 11 or the unintentional provision of different substances from being able to occur simultaneously when substances are repeatedly not removed.

In one particular variant of an embodiment of the invention, which is not shown in FIG. 2, the system can in this way be used for patient monitoring by virtue of its transmitting information about the punctual taking of medicaments to a remote monitoring control center using a data communication link. This allows, by way of example, the clinic or the treating physician to be kept up to date about a patient's therapy compliance and to carry out medicament monitoring. In this case, it is also possible, in particular, for side effects of medicaments, possibly in direct dependence on previously dispensed medicaments, to be monitored.

FIG. 3 schematically shows a variant of an embodiment of the invention which is intended to be used, in particular, for sensory monitoring of patients. The system shown in FIG. 3 is based on a central electronic data processing device 1 which is permanently connected to the other components. To be able to perform sensory monitoring of the patient, a weight sensor 13 is integrated in the vehicle's seat and a respiration rate sensor 14 is integrated in the restraining belt. The weight sensor 13 is built into the seat's support in the form of an acceleration or force sensor. The weight of the person sitting on it is calculated from the difference between the total weight and the weight of the seat without any load. The respiration weight sensor 14 measures the extended length of the restraining belt, which runs over the chest of the person it is belting in and is therefore extended and retracted on the basis of the person's breathing.

Further sensors (which are not shown in FIG. 3) can be provided which relate to the contact between the driver's hands and the steering wheel, to optical scanning of the occupants, to the vehicle seats and to body sensors which need to be applied by the occupants themselves, etc. Possible physiological measurement parameters can include, for example, the body weight using weight sensors in the seat, the body fat and water content using an impedance measurement on the steering wheel, the pulse using a pressure or ECG measurement on the steering wheel, on the restraining belt, on the gear lever or using a pulse oximeter, the respiration rate and depth using the restraining belt, the ECG using a three-point scanning system using the safety belt, the seat position using the seat, the blood oxygen saturation using an optical measurement on the steering wheel, the blood sugar using a contactless measurement on the lens of the eye, the sense of a difficulty in breathing using breathing noises, the ratio of stomach to chest breathing using the safety belt or a laser measurement, a magnetoencephalographic measurement using the steering wheel, gas analyses using an “electric nose”, a cardiotocogram using the safety belt, ultrasound measurements using the safety belt. Electrical scanning points which are possible are all the switches in the motor vehicle, zones on the steering wheel or the gear lever, for example.

Subjective parameters can be recorded by dialog modules assisted by expert systems, to which end the system has at least one interactive communication device. The scope of questions is based on the current state of health of the patient. An intelligent interview system can question the occupant and, by way of example, can obtain subjective information about the general state and the degree of severity of the illness. In addition, GPS data from a GPS sensor as in FIG. 1 can be used to record a long-term profile relating to orientation capabilities, alertness and driving behavior, to which end the system evaluates the dynamics and the uniform nature of the speed and direction of movement.

Useful measurement combinations are obtained from the respective medical indication. By way of example, in the case of diabetes, the body weight would be measured, the body fat and water content, the pressure distribution on the vehicle's seat, the ECG and the breathing rate. In the case of cardiac insufficiency, it would be possible to monitor the body weight, the body fat and water content and the sense of a difficulty in breathing. In the case of asthma, the respiration rate and the sense of a difficulty in breathing could be monitored. In the case of a pregnancy involving some risk, the cardiotocogram, the weight and the ultrasound measurements could be suitable.

To be able to record data for individual patients, the system has an identification sensor 8. Depending on the type of measurement, further, external influencing variables can be necessary in order to interpret the measurement results. These include the vehicle speed, which can be accessed by the system using a link to the vehicle's tachometer 2. In addition, the system can have a distance sensor 15 which measures the vehicle's distance from other vehicles and thus allows conclusions to be drawn about the risk potential of the current driving situation. These data can be used to recognize stress-inducing parameters and situations and to use them to interpret the measured physiological measurement parameters.

The recorded data are firstly stored in the memory in the electronic data processing device 1 and are secondly transmitted to a monitoring control center using the data communication modem 16, which possibly operates using a car telephone which has already been provided. In addition, if the system has GPS information available, then emergency measures can immediately be initiated and directed to the present location of the vehicle in the event of medical emergencies occurring, such as sudden heart failure.

Depending on the clinical picture of the occupants who are to be monitored, the memory in the electronic data processing device 1 has demographic data stored in order to be able to provide the occupants with the relevant information. By way of example, warnings can be given before driving through areas with a flue epidemic, or asthma patients can be warned about zones with increased air pollution.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A motor vehicle, comprising:
assistance means for assisting at least one of a diagnosis, therapy and prophylactic treatment on a person who is in the vehicle;
electronic data processing means for at least one of actuating and interrogating the assistance means; and
at least one of means, activatable by the assistance means, for requesting the person to perform an action, and
identification means, connected to the electronic data processing device, for identifying the person and for at least one of actuating and interrogating the assistance means on the basis of the result of the identification of the person.

2. The motor vehicle as claimed in claim 1, wherein the assistance means includes measuring means for measuring physiological parameters for the person.

3. The motor vehicle as claimed in claim 1, wherein the activatable means comprises a medicaments dispenser for providing medicaments in at least one of solid and liquid forms such that they are removable by the person.

4. The motor vehicle as claimed in claim 1, wherein the activatable means includes communication means for at least one of the output of information to and input of information by the person.

5. The motor vehicle as claimed in claim 1, wherein the electronic data processing device is connected to means for acquiring data about the current road traffic situation and for at least one of actuating and interrogating the assistance means on the basis of these data.

6. The motor vehicle as claimed in claim 1, wherein the electronic data processing device accesses a data communication link in order to interchange data with an electronic patient record.

7. The motor vehicle as claimed in claim 1, wherein the electronic data processing device accesses a data communication link in order to receive information on the basis of an evaluation of the data ascertained by the assistance means.

8. The motor vehicle as claimed in claim 7, wherein the information received is at least one of health-related recommendations and instructions.

9. The motor vehicle as claimed in claim 2, wherein the physiological parameters include at least one of blood pressure and heart rate.

10. The motor vehicle as claimed in claim 2, wherein the activatable means comprises a medicaments dispenser for providing medicaments in at least one of solid and liquid forms such that they are removable by the person.

11. The motor vehicle as claimed in claim 2, wherein the activatable means includes communication means for at least one of the output of information to and input of information by the person.

12. The motor vehicle as claimed in claim 2, wherein the electronic data processing device is connected to means for acquiring data about the current road traffic situation and for at least one of actuating and interrogating the assistance means on the basis of these data.

13. The motor vehicle as claimed in claim 2, wherein the electronic data processing device accesses a data communication link in order to interchange data with an electronic patient record.

14. The motor vehicle as claimed in claim 2, wherein the electronic data processing device accesses a data communication link in order to receive information on the basis of an evaluation of the data ascertained by the assistance means.

15. The motor vehicle as claimed in claim 7, wherein the information received is at least one of physiotherapy exercises and posture exercises.

16. A device for use in a vehicle, comprising:
assistance means for assisting at least one of a diagnosis, therapy and prophylactic treatment on a person in the vehicle;
electronic data processing means for at least one of actuating and interrogating the assistance means; and
at least one of means, activatable by the assistance means, for requesting the person to perform an action, and
identification means, connected to the electronic data processing device, for identifying the person and for at least one of actuating and interrogating the assistance means on the basis of the result of the identification of the person.

17. The device as claimed in claim 16, wherein the assistance means includes measuring means for measuring physiological parameters for the person.

18. The device as claimed in claim 16, wherein the activatable means comprises a medicaments dispenser for providing medicaments in at least one of solid and liquid forms such that they are removable by the person.

19. The device as claimed in claim 16, wherein the activatable means includes communication means for at least one of the output of information to and input of information by the person.

20. The device as claimed in claim 16, wherein the electronic data processing device is connected to means for acquiring data about the current road traffic situation and for at least one of actuating and interrogating the assistance means on the basis of these data.

21. The device as claimed in claim 16, wherein the electronic data processing device accesses a data communication link in order to interchange data with an electronic patient record.

22. The device as claimed in claim 16, wherein the electronic data processing device accesses a data communication link in order to receive information on the basis of an evaluation of the data ascertained by the assistance means.

23. The device as claimed in claim 22, wherein the information received is at least one of health-related recommendations and instructions.

24. The device as claimed in claim 22, wherein the information received is at least one of physiotherapy exercises and posture exercises.
